(19) United States
(12) Patent Application Publication
(10) Pub. No.: US 2024/0416026 A1
(43) Pub. Date: Dec. 19, 2024

(54) MEDICAL SYSTEMS, DEVICES, AND RELATED METHODS FOR WOUND THERAPY

(71) Applicant: Boston Scientific Scimed, Inc., Maple Grove, MN (US)

(72) Inventors: Paul SMITH, Smithfield, RI (US); Jonathan ROOT, Townsend, MA (US); Rahul PRABHU, Cambridge, MA (US); Scott BRECHBIEL, Burlington, MA (US)

(73) Assignee: Boston Scientific Scimed, Inc., Maple Grove, MN (US)

(21) Appl. No.: 18/741,992

(22) Filed: Jun. 13, 2024

Related U.S. Application Data
(60) Provisional application No. 63/508,016, filed on Jun. 14, 2023.

(51) Int. Cl. A61M 1/00

(52) U.S. Cl. CPC A61M 1/916; A61M 1/962; A61M 2205/0216; A61M 2209/08

(57) ABSTRACT

A medical system that includes a handle, a shaft extending distally from the handle, and a cap assembly coupled to the distal end of the shaft. The shaft includes one or more channels extending between the handle and a distal end of the shaft, and the cap assembly includes an opening that is configured to expose the one or more channels at the distal end of the shaft. The cap assembly includes a chamber that extends distally from the opening. The medical system includes a porous body movably disposed within the chamber. The porous body is configured to transition from a compressed configuration when disposed inside the chamber to an expanded configuration upon extending outwardly from the chamber.

### CROSS-REFERENCE TO RELATED APPLICATIONS

This application claims the benefit of priority of U.S. Provisional Patent Application No. 63/508,016, filed on Jun. 14, 2023, the entirety of which is incorporated herein by reference.

### TECHNICAL FIELD

Various aspects of the disclosure generally relate to medical systems, devices, and related methods that may be used to treat a subject. In particular, aspects of the disclosure relate to medical systems, devices, and methods for wound therapy, such as endoscopic vacuum therapy that includes applying negative air pressure to tissue for wound therapy.

### BACKGROUND

Endoscopic and open surgical procedures of the gastrointestinal (GI) tract include, for example, colonic resection, bariatric surgery, esophagectomy, gastric bypass, and sleeve gastrectomy, among others. These procedures may result in perforation, post-surgical leaks, or other wounds of the GI tract. Limited treatment options exist for managing such wounds, which have significant morbidity and mortality rates. Options include surgical re-operation and endoscopic placement of a stent or one or more clips. Surgery is invasive and involves high morbidity and mortality rates. Although endoscopic stent placement is less invasive, the placed stent can migrate from the intended location and/or wall off an infection at the treatment site, which may exacerbate the infection and/or inhibit drainage. The systems, devices, and methods of the current disclosure may rectify one or more of the deficiencies described above or address other aspects of the art.

### SUMMARY

Each of the aspects disclosed herein may include one or more of the features described in connection with any of the other disclosed aspects.

Aspects of the disclosure relate to, among other things, systems, devices, and methods for treating a subject. Aspects of this disclosure relate to medical systems, devices, and methods for wound therapy, such as endoscopic vacuum therapy that includes applying negative air pressure to tissue for wound therapy.

According to an example, a medical system includes a handle; a shaft extending distally from the handle, the shaft including one or more channels extending between the handle and a distal end of the shaft; a cap assembly coupled to the distal end of the shaft, the cap assembly including an opening that is configured to expose the one or more channels at the distal end of the shaft, and a chamber that extends distally from the opening; and a porous body movably disposed within the chamber, the porous body is configured to transition from a compressed configuration when disposed inside the chamber to an expanded configuration upon extending outwardly from the chamber.

Any of the medical systems described herein may include any of the following features. A tube movably coupled to the cap assembly, the tube is configured to move between a first position and a second position relative to the cap assembly to transition the porous body between the compressed configuration and the expanded configuration. In the first position, a distal end of the tube is positioned outside the chamber such that the porous body is maintained inside the chamber and in the compressed configuration; and in the second position, the distal end of the tube is positioned inside the chamber such that the porous body is extended outside of the chamber and transitioned to the expanded configuration. The tube is a vacuum tube that is coupled to a negative pressure source at a proximal portion of the vacuum tube, and to the porous body at a distal portion of the vacuum tube. The vacuum tube is configured to apply negative pressure to the porous body as provided by the negative pressure source. The chamber is configured to receive the tube when the tube is in the second position, and at least a portion of the chamber is deformable to facilitate releasing the tube from the chamber. The chamber is configured to receive the tube when the tube is in the second position, and the chamber includes a slot that is configured to release the tube from the chamber. The cap assembly is removably mounted on an exterior surface of the distal end of the shaft. The cap assembly is selectively rotatable about the exterior surface of the distal end of the shaft such that the chamber is repositionable relative to the one or more channels. The porous body is a sponge, a gauze, a film, or a membrane. The chamber is at least partially transparent such that the porous body disposed inside the chamber is visible through the chamber. The chamber includes a window such that the porous body disposed inside the chamber is visible through the window by an imaging device. At least one of the one or more channels includes a working channel, and the window is aligned with the working channel to allow access to the working channel at the distal end of the shaft. The cap assembly includes a body with a pair of opposing halves, the body is configured to receive the distal end of the shaft and the pair of opposing halves is configured to grasp an exterior of the distal end to couple the cap assembly to the shaft. The cap assembly includes a fastener that is configured to couple the pair of opposing halves to one another, thereby securely attaching the cap assembly to the shaft.

According to another example, a medical device includes a cap assembly configured to be attached about a shaft of an endoscope such that a working channel of the endoscope is accessible through an opening of the cap assembly at a distal end of the shaft, the cap assembly including a chamber that extends distally from the distal end; and a porous body received within the chamber, the chamber configured to compress the porous body relative to the shaft; wherein the porous body is movable relative to the cap assembly from a first position inside the chamber and in a compressed configuration to a second position outside of the chamber and in an expanded configuration.

Any of the medical devices described herein may include any of the following features. tube at least partially disposed inside the cap assembly, the tube is coupled to a negative pressure source at a first end and to the porous body at a second end, such that the negative pressure source is in fluid communication with the porous body via the tube; wherein the tube is movable relative to the cap assembly to extend the porous body out from the chamber, and configured to generate a vacuum through the porous body in response to activating the negative pressure source. The chamber is configured to receive the tube when the tube extends the porous body out from the chamber, and at least a portion of the chamber is deformable to facilitate releasing the tube from the chamber. The chamber is configured to receive the tube when the tube extends the porous body out from the chamber, and the chamber includes a slot that is configured to release the tube from the chamber.

According to another example, a method for treating a wound cavity with a medical device includes positioning a shaft of the medical device at the wound cavity, wherein a cap assembly is mounted onto the shaft such that the cap assembly is positioned adjacent to the wound cavity; extending a porous body out from within the cap assembly in response to moving a tube relative to the cap assembly, such that the porous body expands upon exiting the cap assembly and entering the wound cavity, wherein the tube is coupled to the porous body and in fluid communication with a negative pressure source; and applying a negative pressure at the wound cavity via the porous body in response to activating the negative pressure source.

It may be understood that both the foregoing general description and the following detailed description are exemplary and explanatory only and are not restrictive of the invention, as claimed. As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from a user/toward a treatment site, and the term “proximal” refers to a direction toward a user. The terms “downward,” “upward,” “lower,” “upper,” “bottom,” and “top” may refer to directions relative to the views of the elements shown throughout the drawings. The term “exemplary” is used in the sense of “example,” rather than “ideal.” The term “approximately,” or like terms (e.g., “substantially”), includes values+/−10% of a stated value.

### BRIEF DESCRIPTION OF THE DRAWINGS

The accompanying drawings, which are incorporated in and constitute a part of this specification, illustrate aspects of this disclosure and together with the description, serve to explain the principles of the disclosure.

FIG. 1 shows a perspective view of an exemplary medical system, according to some embodiments.

FIG. 2 shows a perspective view of an exemplary medical device coupled to the medical system of FIG. 1, according to some embodiments.

FIG. 3 shows a top view of the medical device of FIG. 2, according to some embodiments.

FIG. 4A shows a perspective view of the medical device of FIG. 2 in a first position, according to some embodiments.

FIG. 4B shows a perspective view of the medical device of FIG. 2 in a second position, according to some embodiments.

FIG. 4C shows a perspective view of the medical device of FIG. 2 in a third position, according to some embodiments.

FIG. 4D shows a perspective view of the medical device of FIG. 2 in a fourth position, according to some embodiments.

FIG. 5 shows a front elevational view of another exemplary medical device coupled to the medical system of FIG. 1, according to some embodiments.

FIG. 6 shows a front elevational view of the medical device of FIG. 5 in an alternative configuration, according to some embodiments.

### DETAILED DESCRIPTION

Endoluminal vacuum therapy (EVAC) is an adaptation of negative pressure wound therapy (i.e., vacuum therapy or wound vac), which may be used for external treatment of chronic, non-healing wounds, where a vacuum-sealed material (e.g., a sponge) is inserted into the wound and a negative pressure is applied to the sponge to promote drainage. In a typical EVAC procedure, negative pressure is delivered to a wound site internally within the GI tract, for example through a nasogastric tube having a sponge at its terminal end. The sponge may be placed endoscopically into a perforation, leak, or other wound, and negative pressure may then be applied to promote drainage from the wound.

Embodiments of this disclosure include devices, systems, and methods specifically for EVAC procedures. In some embodiments, EVAC may include endoluminal placement of a porous body (e.g., a sponge or other like material) into a wound site, for example a perforation, a cyst, a leak, or an anastomosis. The porous body may be placed within a wound via a catheter, scope (e.g., endoscope, bronchoscope, colonoscope, etc.), tube, or sheath, which may be inserted into the GI tract via a natural orifice. The orifice may be, for example, the nose, mouth, or anus, and a distal end of the catheter, scope, tube, or sheath (and thus the porous body) may be positioned in any portion of the GI tract, including the esophagus, stomach, duodenum, large intestine, or small intestine.

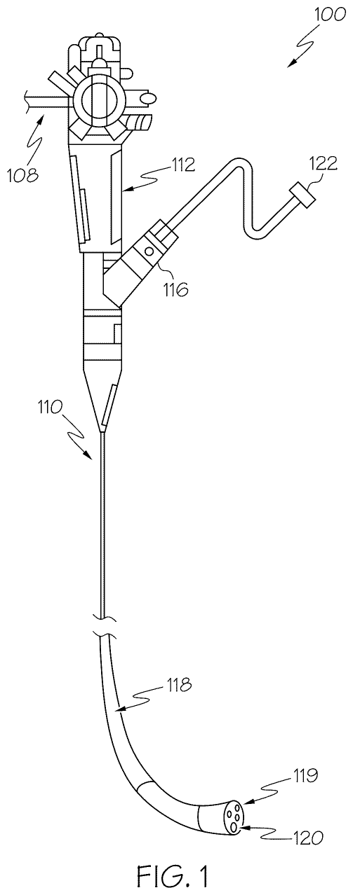

FIG. 1 depicts an exemplary medical system 100. Medical system 100 may include an insertion device, such as an endoscope, which may be inserted into an esophagus of a patient. Medical system 100 may include a handle 112 and a shaft 110 extending distally from handle 112. Shaft 110 may include one or more channels extending therethrough from a proximal portion positioned adjacent to handle 112 and a distal portion 118 terminating at a distal tip 119. In some embodiments, medical system 100 may include an umbilicus (not shown), which may connect a port 108 of the endoscope to sources of, for example, air, water, suction, power, image processing and/or viewing equipment. In some embodiments, medical system 100 may include an imaging element and/or a lighting element, such as at distal tip 119, to aid in accurately positioning shaft 110 adjacent a target treatment site (e.g., a wound cavity) during an EVAC procedure. Using one or more of the channels of shaft 110, such as working channel 120, a user of medical system 100 may deploy or otherwise deliver a medical tool or instrument to the target treatment site, such as a medical tool 122 received through a port 116 on handle 112.

In some aspects, handle 112 may include one or more actuators along a proximal end of handle 112 (e.g., adjacent to port 108), for example, to control the movement of shaft 110, and particularly distal portion 118, the activation of one or more imaging element(s) and lighting element(s), and control a deflection, position, or orientation of distal tip 119. It is noted that FIG. 1 illustrates distal tip 119 of medical system 100 (e.g., an endoscope) as being “forward-facing” in that the features of distal tip 119, such as the one or more channels of shaft 110, may face distally (i.e., forward of a distalmost face of distal tip 119). It should be appreciated that this disclosure also encompasses other configurations of distal tip 119, including distal tip 119 being “side-facing”in which the one or more channels of shaft 110 may be disposed on a radially outer side of distal tip 119 so that they point in a radially outward direction, approximately perpendicularly to a longitudinal axis of distal portion 118.

Still referring to FIG. 1, although insertion device or medical system 100 is discussed above as being an endoscope, this disclosure is not so limited. Although the disclosure may refer at different points to an endoscope, it will be appreciated that, unless otherwise specified, duodenoscopes, endoscopes, gastroscopes, endoscopic ultrasonography (“EUS”) scopes, colonoscopes, ureteroscopes, bronchoscopes, laparoscopes, cytoscopes, aspiration scopes, sheaths, catheters, or any other suitable delivery device or insertion device may be used in connection with the systems, devices, elements, assemblies, methods, etc. described herein.

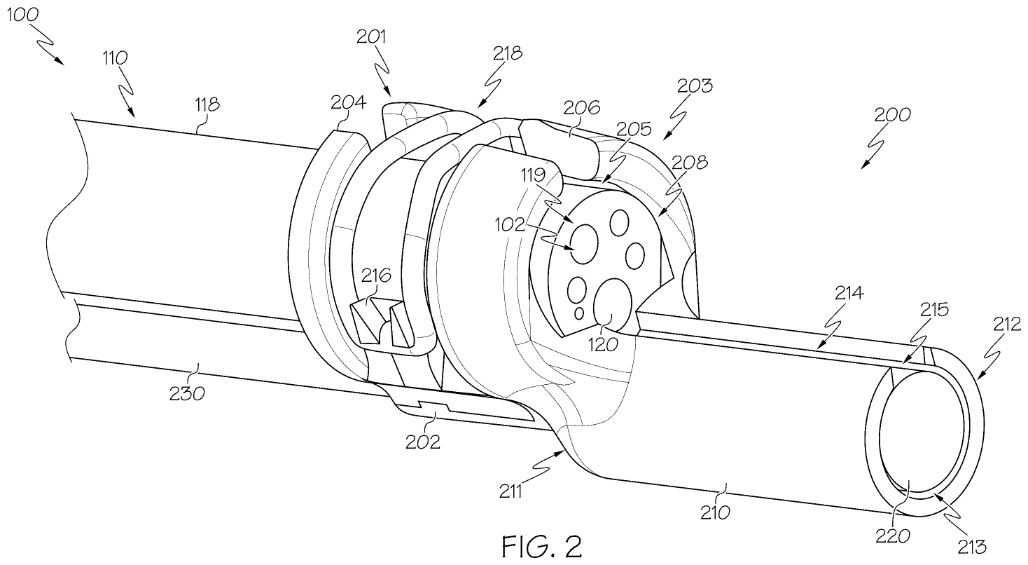

Referring now to FIG. 2, medical system 100 may include a medical device coupled to shaft 110. In the example, the medical device may include a cap assembly 200 that may be removably coupled to shaft 110 along distal portion 118. In other words, cap assembly 200 may be removably mounted onto distal portion 118. Cap assembly 200 may be configured to attach to an exterior surface of shaft 110 adjacent to distal tip 119 via various suitable means. In one example, cap assembly 200 may form a frictional engagement with the exterior surface of shaft 110. In embodiments in which shaft 110 includes an articulating section, it should be appreciated that cap assembly 200 may be coupled to shaft 110 distally relative to the articulating section. For example, an entirety of cap assembly 200 may be distal to a distalmost end of the articulation section.

Cap assembly 200 may include a body 202 defined by a proximal end 201 and a distal end 203. In the example, body 202 may have a cylindrical configuration that is sized, shaped, and/or otherwise configured to receive shaft 110. In other words, body 202 may define a channel 205 extending through cap assembly 200, with body 202 being configured to receive shaft 110 through channel 205 such that body 202 may extend about the exterior surface of shaft 110, and particularly distal portion 118, when shaft 110 is received therethrough. Cap assembly 200 may be configured such that an interior surface of body 202 that defines channel 205 may frictionally engage distal portion 118 of shaft 110 to inhibit movement (e.g., axial translation) of distal tip 119 received therein. Accordingly, body 202 may be configured to engage and/or grasp an exterior of distal portion 118 when distal portion 118 is received through body 202, thereby coupling cap assembly 200 to shaft 110. Body 202 may define an opening 208 adjacent to distal end 203 for receiving distal tip 119 of shaft 110 when distal portion 118 is received through channel 205 of body 202.

In some embodiments, proximal end 201 and distal end 203 may include a pair of adjustable rings 204, 206 that are each configured to move relative to one another to facilitate engagement with shaft 110. For example, a proximal adjustable ring 204 may be defined at proximal end 201 and a distal adjustable ring 206 may be defined at distal end 203. Each of the adjustable rings 204, 206 may be selectively adjustable to receive distal portion 118 through body 202 based on a cross-sectional dimension (e.g., a diameter) of shaft 110. Stated differently, the adjustable rings 204, 206 at the opposing ends 201, 203 may define a pair of opposing flexible halves of body 202 that are each independently configured to partially flex laterally inward and/or outward to accommodate shafts of varying cross-sectional dimensions. Accordingly, proximal end 201 and distal end 203 may be configured to engage and/or grasp an exterior of distal portion 118 when distal portion 118 is received through body 202, thereby coupling cap assembly 200 to shaft 110.

Still referring to FIG. 2, cap assembly 200 may include a fastening mechanism for securely attaching body 202 to shaft 110. The fastening mechanism may include various suitable devices for attaching body 202 to shaft 110, including a clip, an elastic band, an O-ring, a clamp, and more. In the example shown in FIG. 2, the fastening mechanism may include a pair of protrusions 216 disposed along opposing sides of body 202 and a flexible fastener 218 with opposing ends that may be configured to engage each of the pair of protrusions 216. In this instance, flexible fastener 218 may be configured to couple the pair of opposing halves of body 202 (e.g., proximal end 201 and distal end 203) to one another with distal portion 118 received therethrough, thereby securely attaching body 202 to shaft 110.

Cap assembly 200 may further include a chamber 210 extending distally from body 202, and more particularly from distal end 203. Chamber 210 may have a longitudinal length defined between a first (proximal) end 211 positioned at distal end 203 and a second (distal) end 212 positioned opposite first end 211. In the example, chamber 210 may have a substantially rigid configuration. For example, chamber 210 may be formed of a thermoplastic polymer, a semi-rigid plastic, etc. In other examples, chamber 210 may include a pocket, a bag, or other suitable components with a relatively flexible configuration.

Still referring to FIG. 2, chamber 210 may define an interior lumen 215 that is sized, shaped, and/or otherwise configured to receive one or more vacuum therapy devices therein. For example, the vacuum therapy device may include a device that is configured to interface with a target treatment site (e.g., tissue) within a subject (e.g., a patient). The vacuum therapy device may be at least partially absorbent and include, but is not limited to, a vacuum therapy sponge, a gauze, a film, a membrane, and more. In the example, the vacuum therapy device may include a porous body 220. In some embodiments, porous body 220 may include any suitable biocompatible material that may absorb liquids and/or permit liquid to pass therethrough via negative pressure. The material may be flexible, compressible, porous, hydrophilic, sterile, and/or disposable. The material of porous body 220 may be or may include an open-cell foam. Suitable materials may include polyurethanes, polymers with ester and/or ether functional groups, composite materials, and any other medical-grade material or materials.

Porous body 220 may have a longitudinal length and a lateral diameter and/or width that are at least partially flexible. As such, porous body 220 may be at least partially compressible such that chamber 210 may be configured to compress porous body 220 to a compressed configuration when received therein. Chamber 210 may be further configured to (axially) fix porous body 220 therein absent an application of force applied to porous body 220 for deployment from chamber 210.

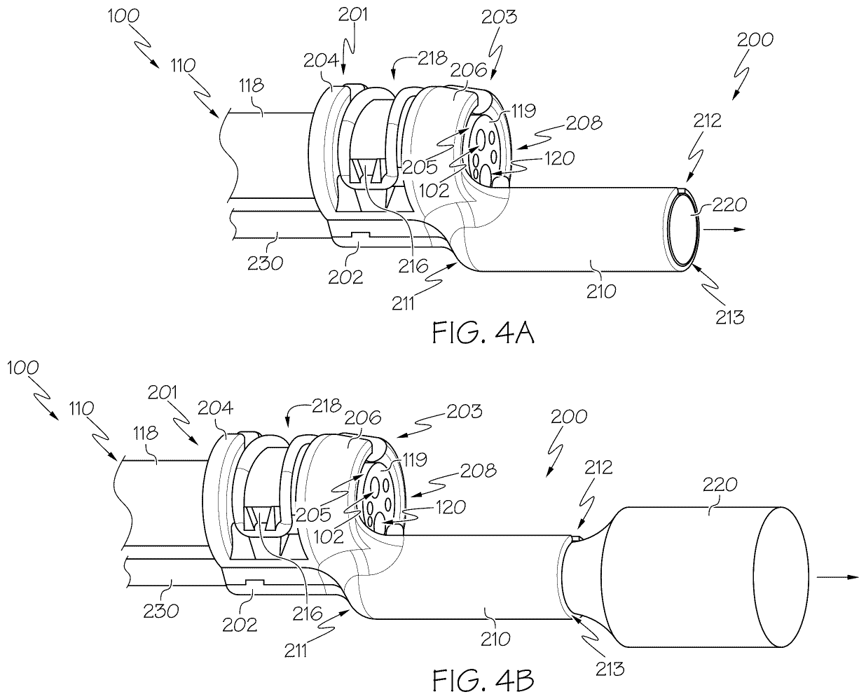

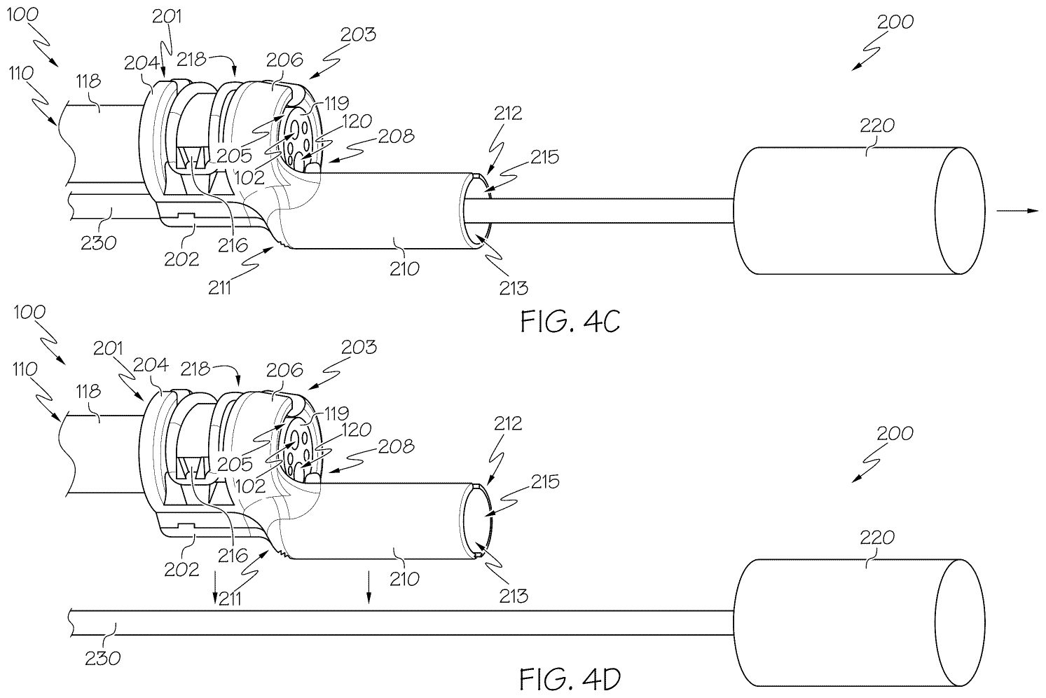

It should be appreciated that a diameter of porous body 220 may correspond to a diameter of interior lumen 215 of chamber 210 when in the compressed configuration. For example, the diameter of interior lumen 215 may be less than about 20 millimeters to facilitate navigation of cap assembly 200 through a tortuous pathway of the subject (e.g., a patient), such as an esophagus. As described further herein, cap assembly 200 may be configured to transition porous body 220 from the compressed configuration to an expanded configuration upon deploying porous body 220 distally from interior lumen 215 of chamber 210 (see FIGS. 4B-4D).

Chamber 210 may include a distal opening 213 at second end 212 that is sized and/or shaped to facilitate deployment of porous body 220 from within chamber 210. Chamber 210 may further include a window 214 extending along the longitudinal length of chamber 210, and particularly between first end 211 and second end 212. Window 214 may be, for example, a slot. With porous body 220 received within chamber 210, cap assembly 200 may be operable to facilitate visualization of porous body 220 from an exterior of chamber 210 via window 214. In other words, cap assembly 200 may be operable to provide visual feedback of a location of porous body 220 during a procedure of delivering porous body 220 to a target treatment site (e.g., a wound cavity) via medical system 100 and cap assembly 200 through window 214. For example, chamber 210 may be positioned relative to distal tip 119 such that an imaging assembly 102 of medical system 100 may be operable to visualize porous body 220 through window 214. In other examples, window 214 may be omitted entirely. In some embodiments, chamber 210 may be formed of an opaque material. In other embodiments, at least a portion of chamber 210 may be transparent and/or semi-transparent to further facilitate visual inspection of porous body 220 from within chamber 210. In this instance, porous body 220 may be visible through chamber 210.

Still referring to FIG. 2, cap assembly 200 may include a fluidics component that is coupled to, and in fluid communication with, porous body 220. For example, the fluidics component of cap assembly 200 may include a tube member 230 (e.g., a vacuum tube) that is coupled to a proximal end of porous body 220. Tube member 230 may be formed from a polymer or any other suitable biocompatible material. In some embodiments, tube member 230 may include a shape memory membrane, for example a Nitinol membrane, or may be formed of a shape memory and/or heat-set material (e.g., Nitinol). As described herein, tube member 230 may be configured to move porous body 220 in response to movement of tube member 230 relative to body 202. In the example, tube member 230 may be positioned outside of shaft 110 (e.g., along a radially outer side of shaft 110), and may extend into body 202 at proximal end 201. When porous body 220 is disposed within chamber 210, a proximal end of porous body 220 may be positioned adjacent to distal end 203 such that a terminal (distal) end of tube member 230 may similarly be positioned adjacent to distal end 203 and disposed relatively proximal to first end 211. Tube member 230 may be configured to move (e.g., translate) porous body 220 relative to chamber 210, such as in a distal direction through distal opening 213 to extend porous body 220 outwardly from chamber 210, in response to tube member 230 moving distally relative to body 202 (see FIGS. 4B-4D). In this instance, tube member 230 may extend distally relative to first end 211 and into chamber 210.

Although not shown, it should be understood that tube member 230 may include one or more openings and/or ports at a distal end of tube member 230, such that the one or more openings and/or ports are fluidly coupled to porous body 220. As described herein, tube member 230 may be fluidly coupled to a negative pressure (vacuum) source at a proximal end of tube member 230 (not shown) that is opposite of the distal end of tube member 230. Accordingly, porous body 220 may be in fluid communication with the negative pressure source via tube member 230, and particularly through the one or more openings and/or ports at the distal end of tube member 230.

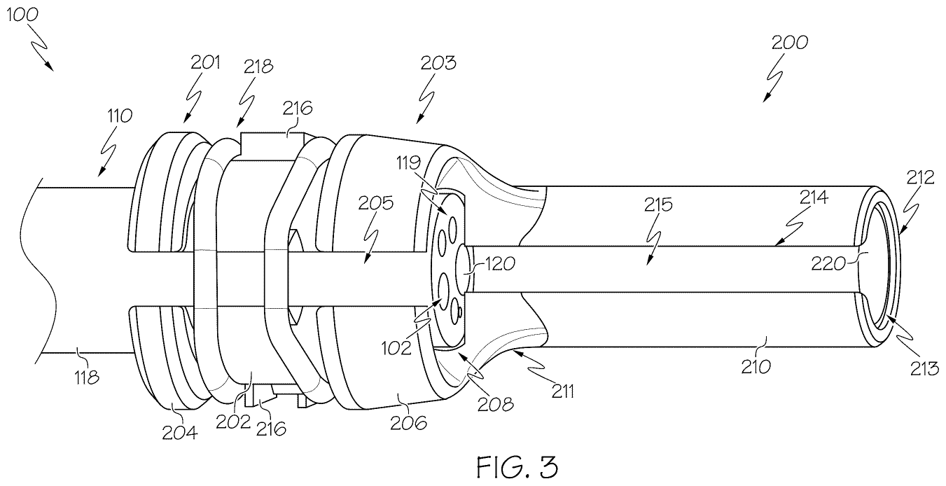

Referring now to FIG. 3, body 202 may be sized, shaped, and/or otherwise configured to position chamber 210 relatively underneath (e.g., radially outward of) and/or axially offset from the one or more channels of shaft 110, including working channel 120, when cap assembly 200 is coupled to distal portion 118. Stated differently, chamber 210 may be arranged relative to body 202 such that chamber 210 is suspended below an opening of the one or more channels of shaft 110 to prevent any portion of cap assembly 200 from blocking the channels, including working channel 120, when body 202 is coupled to distal portion 118. In this instance, an opening of working channel 120 and the other channels of shaft 110 may be accessible at distal tip 119 via opening 208, such that one or more medical tools or instruments (e.g., air, water, light, etc.) may extend through said channels and out of shaft 110 for accessing the target treatment site via opening 208. In some embodiments, working channel 120 may be at least partially aligned with window 214 such that a medical tool or instrument exiting working channel 120 may extend outwardly from cap assembly 200 through window 214.

In exemplary use, as shown in FIGS. 4A-4D, medical system 100 may be utilized to perform endoluminal vacuum therapy to treat a target treatment site by deploying a vacuum therapy device (e.g., porous body 220) via cap assembly 200. For example, medical system 100 may be operated such that shaft 110 is navigated through a subject (e.g., a patient), such as within the subject's GI tract, until arriving at the target treatment site (e.g., a wound cavity). The wound cavity may be in the form of an anastomotic leak, a perforation, or other injury within the GI tract. Distal tip 119, with cap assembly 200 coupled thereto, may be positioned adjacent, through, and/or inside the wound cavity. It should be appreciated that with cap assembly 200 coupled to distal tip 119, enhanced positional control is provided in installing porous body 220 relative to the would cavity due to the maneuverability capabilities of shaft 110, such as through control of the one or more actuators on handle 112. A location of porous body 220 may be determined based on the visual feedback generated by cap assembly 200 by visualizing the position of porous body 220 through window 214 (e.g., with imaging assembly 102) while porous body 220 remains disposed inside chamber 210.

Referring specifically to FIG. 4A, distal tip 119 may be arranged to position chamber 210 towards the wound cavity. In this instance, porous body 220 may access the wound cavity, in addition to any medical tools or instruments disposed in the one or more channels of shaft 110 which are accessible through cap assembly 200 via opening 208. In this instance, tube member 230 may be in a first position with porous body 220 disposed entirely within chamber 210. It should be appreciated that the first position of tube member 230 is a proximalmost position in which tube member 230 is positioned outside of chamber 210. In the first position, a distal end of tube member 230 may be positioned outside of chamber 210. In other embodiments, porous body 220 may have a longitudinal length that is smaller than a longitudinal length of chamber 210. In this instance when in the first position, the distal end of tube member 230 may be positioned outside of (proximal to) chamber 210 with a distal end of porous body 220 positioned proximal to distal end 212 of chamber 210, or inside of chamber 210 with the distal end of porous body 220 positioned flush with distal end 212. Tube member 230 may be moved from the first position towards a second position to initiate deployment of porous body 220 distally from within chamber 210.

As seen in FIG. 4B, distal movement (e.g., translation) of tube member 230 towards the second position may provide a corresponding movement of porous body 220, thereby causing distal portions of porous body 220 to exit chamber 210 via distal opening 213. In response to moving towards the second position, tube member 230 may extend into chamber 210 such that at least a portion of tube member 230 and at least a portion of porous body 220 are simultaneously positioned inside chamber 210. As porous body 220 extends out from chamber 210, the laterally/radially inward forces applied by chamber 210 onto an exterior of porous body 220 are gradually removed such that porous body 220 is permitted to expand. Stated differently, tube member 230 may be configured to transition porous body 220 from the compressed configuration (FIG. 4A) to the expanded configuration as porous body 220 is pushed distally out of chamber 210 when tube member 230 moves from the first position to the second position.

Referring now to FIG. 4C, continued distal movement of tube member 230 to a third position may cause porous body 220 to completely exit chamber 210, thereby transitioning an entirety of porous body 220 to the expanded configuration. It should be appreciated that porous body 220 may expand in a lateral/radial direction, an axial direction, and/or various other suitable manners when transitioning to the expanded configuration. In some embodiments, a position of porous body 220 may be controlled via medical system 100, and particularly by moving shaft 110. In further embodiments, a position of porous body 220 may be controlled via movement of tube member 230 relative to shaft 110, body 202, and chamber 210.

As briefly described above, tube member 230 may be fluidly coupled to a negative pressure source at a proximal end of tube member 230 (not shown) that is opposite of the terminal (distal) end of tube member 230 coupled to porous body 220. Accordingly, porous body 220 may be in fluid communication with the negative pressure source via tube member 230. As such, a negative pressure may be generated at porous body 220 via tube member 230, in response to activating the negative pressure source, to generate a suction around a surrounding environment of porous body 220, and particularly at the wound cavity.

Upon positioning porous body 220 at the wound cavity, the negative pressure source fluidly coupled to tube member 230 may be activated to apply the negative pressure to the wound cavity through porous body 220. In some aspects, fluid within a wound cavity may flow into porous body 220 and through tube member 230, for example, via capillary action, with negative pressure being applied through tube member 230. In this instance, porous body 220 may be configured to treat the wound cavity by suctioning any material (e.g., fluid) therein into the porous body 220 and through tube member 230, such as fluids from a post-surgical leak or perforation for purposes of preventing drainage and promoting healing of the wound cavity.

In other words, medical system 100 (with cap assembly 200 coupled thereto) may be operable to perform endoluminal vacuum therapy with cap assembly 200 facilitating deployment of porous body 220 (e.g., a vacuum sealed sponge) to the wound cavity for performing negative pressure wound therapy. Accordingly, cap assembly 200 (and particularly porous body 220) may help to ensure that drainage of fluid is maintained as the wound cavity decreases in size throughout the healing process. In some aspects, tube member 230 and porous body 220 may also be used to deliver fluid (e.g., saline, an antibiotic fluid, etc.) to the wound cavity, for example, to aid in the flushing and/or otherwise treating the wound cavity.

In some embodiments, tube member 230 and porous body 220 may be decoupled from body 202 and chamber 210, such as for purposes of removing shaft 110 from the subject while maintaining porous body 220 at the wound cavity. In the example, body 202 and/or chamber 210 may be formed of a material that is frangible and/or otherwise configured to at least partially deform upon an application of a predetermined force thereto, thereby releasing tube member 230 from chamber 210. Stated differently, a force exceeding a predetermined threshold may be applied to body 202 and/or chamber 210 by tube member 230 moving to a fourth position, such as against an interior surface of body 202 and/or chamber 210, thereby causing body 202 and/or chamber 210 to permanently deform for releasing tube member 230 from within chamber 210.

For example, as seen in FIG. 4D, tube member 230 may be moved relatively downward/radially outward to the fourth position until body 202 and/or chamber 210 is deformed along their respective lower surfaces such that openings are formed to allow tube member 230 to move out from body 202 and chamber 210. In this instance, shaft 110 may be removed entirely from the subject while tube member 230 and porous body 220 may be controlled independently from medical system 100. It should be appreciated that the expanded configuration of porous body 220 may provide an increased surface area of porous body 220 in the wound cavity, which may help to allow porous body 220 to collect (e.g., absorb) more fluid from the wound cavity than a typical cylindrical sponge. Additionally, the increased surface area and increased absorbency may help to allow for medical system 100 (e.g., including porous body 220) to remain within the wound cavity for a longer period of time (i.e., with fewer removals and/or replacements).

As the wound cavity decreases in size throughout the healing process, a physician, or other user, may manually remove porous body 220 from the wound cavity for replacement with a subsequent porous body with a smaller cross-sectional dimension. In this instance, medical system 100 may be equipped with another cap assembly 200 onto shaft 110 for subsequent deployment of the smaller porous body 220 at the wound cavity. In the example, a relatively smaller porous body 220 may be continuously applied to the wound cavity via medical system 100 at regular intervals for promoting treatment of the wound. One or more of porous body 220 or tube member 230 may have antiseptic properties, for example, to help prevent or inhibit infection and/or prolong the period for which porous body 220 may remain within the wound cavity.

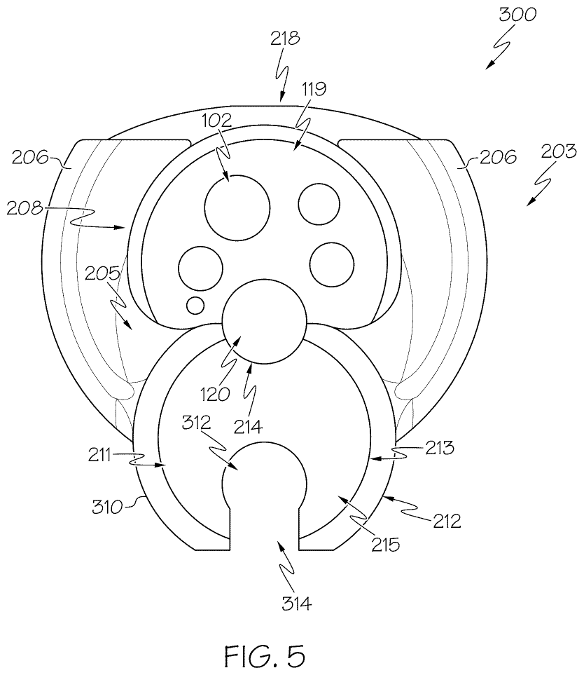

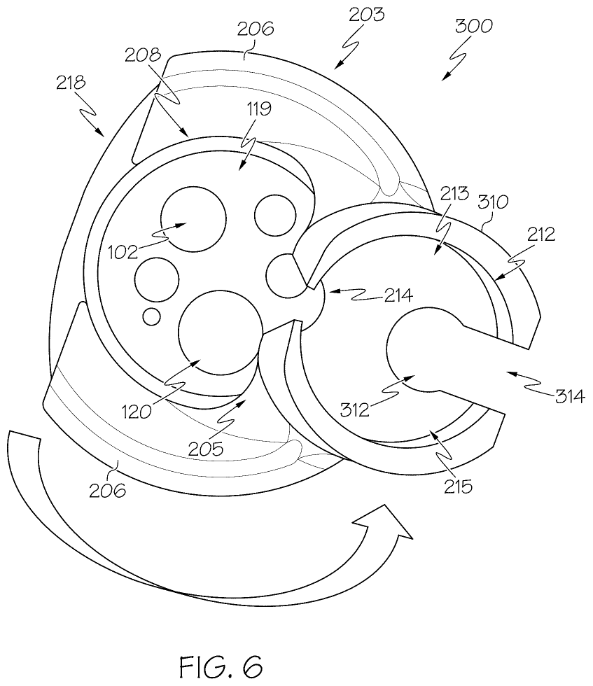

Referring now to FIGS. 5-6, another exemplary cap assembly 300 is depicted. Cap assembly 300 may be substantially similar to cap assembly 200 except for the differences explicitly described herein. Accordingly, the same reference numerals are used to identify substantially similar components. Additionally, cap assembly 300 may be configured and operable similar to cap assembly 200. Referring specifically to FIG. 5, cap assembly 300 may include a chamber 310 with an opening 312 formed within first (proximal) end 211. Opening 312 may be sized, shaped, and/or otherwise configured to receive tube member 230 through first (proximal) end 211 and into chamber 310. In some embodiments, opening 312 may be configured to removably anchor tube member 230 to chamber 310. In the example, opening 312 may be substantially C-shaped in accordance with a circular and/or cylindrical cross-sectional configuration of tube member 230. It should be appreciated that cap assembly 200 may similarly include opening 312 at first (proximal) end 211 for facilitating receipt of tube member 230 within chamber 210.

In the example, chamber 310 may include a slot and/or a notch 314 that defines a second opening along first (proximal) end 211. Opening 312 and notch 314 may collectively define a continuous void within first (proximal) end 211 in which notch 314 extends downwardly from opening 312 and towards a bottom surface of chamber 310. Notch 314 may form an opening along an outer perimeter of chamber 310 for permitting removal of tube member 230 from chamber 310. In the example, notch 314 may define a cross-sectional void that is relatively smaller than opening 312 such that tube member 230 is at least partially inhibited from exiting opening 312 and entering notch 314 absent an application of force onto tube member 230.

Still referring to FIG. 5, notch 314 may be configured to facilitate removal of tube member 230 from within chamber 310 (and laterally/radial out of opening 312) without deforming cap assembly 300 in response to applying a downward force onto tube member 230 in a direction towards notch 314. Thus, pushing tube member 230 relatively downward from opening 312 may allow tube member 230 to decouple from chamber 310 by exiting through notch 314. It should be appreciated that although opening 312 and/or notch 314 are shown and described herein as being positioned proximate to a bottom surface of first (proximal) end 211 and/or chamber 310, one or more of opening 312 and/or notch 314 may be positioned along various other surfaces or portions of first (proximal) end 211 and/or chamber 310.

In some embodiments, tube member 230 may further include a guidewire, an outer sheath, an inner sheath, a stiffening mandrel, or other suitable devices. In one example, tube member 230 may be flexible and disposed within a rigid outer sheath (not shown) that is configured to enhance a rigidity of tube member 230 for purposes of deploying porous body 220 from chamber 310. In other words, the guidewire, outer sheath, inner sheath, and/or stiffening mandrel may be configured to inhibit tube member 230 from bending and/or kinking. The outer sheath may include a removable portion that is configured to peel and/or cut away from a remainder portion of the outer sheath to facilitate removal of tube member 230. In other examples, tube member 230 may include a guidewire and/or a stiffening mandrel (not shown) disposed therein for enhancing a rigidity of tube member 230 for purposes of deploying porous body 220 from chamber 310. In this instance, the guidewire and/or a stiffening mandrel may define a rail for facilitating movement of tube member 230 along said rail. In further examples, both tube member 230 and at least one of a guidewire and/or a stiffening mandrel may be disposed within an outer sheath. It should be appreciated that various other suitable configurations of devices may be coupled to and/or assembled with tube member 230.

Referring now to FIG. 6, cap assembly 300 may be configured to selectively move (e.g., rotate) relative to distal tip 119 of shaft 110. In this instance, cap assembly 300 may be movably coupled to distal tip 119 such that an arrangement and/or orientation of body 202 may be adjusted relative to distal tip 119. By adjusting cap assembly 300 relative to shaft 110, opening 208 and chamber 310 may be repositioned to various suitable configurations relative to the one or more channels of shaft 110, and particularly the openings of said channels at distal tip 119. Accordingly, chamber 310 (and porous body 220 received therein) may be aligned adjacent to particular channels of shaft 110, and more specifically to particular medical tools and/or instruments disposed within said channels, to facilitate access for said medical tools and/or instruments to porous body 220. In this instance, a relative position of chamber 310 to the distal end openings of the channels at distal tip 119 may provide enhanced accessibility of porous body 220 to the one or more medical tools and/or instruments received within said channels of shaft 110 to improve control of porous body 220 during a procedure.

In some embodiments, shaft 110 may include one or more markings (e.g., arrows) along distal portion 118 and/or distal tip 119 to facilitate various suitable arrangements of cap assembly 300. Body 202 of cap assembly 300 may similarly include corresponding markings for alignment with the markings on shaft 110 to further facilitate visual alignment of the complimentary devices.

While principles of this disclosure are described herein with reference to illustrative examples for particular applications, it should be understood that the disclosure is not limited thereto. For example, the disclosure refers to EVAC as an exemplary procedure, and the GI tract as a typical lumen for the systems and methods of the disclosure. The systems, devices, and methods of the present disclosure, however, may be used in any suitable medical procedure in any lumen or cavity within the body, for example, to aid in drainage from a wound anywhere within the body. Those having ordinary skill in the art and access to the teachings provided herein will recognize additional modifications, applications, and substitution of equivalents all fall within the scope of the examples described herein. Accordingly, the invention is not to be considered as limited by the foregoing description.

What is claimed is:

1. A medical system comprising:
a handle;
a shaft extending distally from the handle, the shaft including one or more channels extending between the handle and a distal end of the shaft;
a cap assembly coupled to the distal end of the shaft, the cap assembly including an opening that is configured to expose the one or more channels at the distal end of the shaft, and a chamber that extends distally from the opening; and
a porous body movably disposed within the chamber, the porous body is configured to transition from a compressed configuration when disposed inside the chamber to an expanded configuration upon extending outwardly from the chamber.

2. The medical system of claim 1, further comprising a tube movably coupled to the cap assembly, the tube is configured to move between a first position and a second position relative to the cap assembly to transition the porous body between the compressed configuration and the expanded configuration.

3. The medical system of claim 2, wherein, in the first position, a distal end of the tube is positioned outside the chamber such that the porous body is maintained inside the chamber and in the compressed configuration; and
wherein, in the second position, the distal end of the tube is positioned inside the chamber such that the porous body is extended outside of the chamber and transitioned to the expanded configuration.

4. The medical system of claim 3, wherein the tube is a vacuum tube that is coupled to a negative pressure source at a proximal portion of the vacuum tube, and to the porous body at a distal portion of the vacuum tube.

5. The medical system of claim 4, wherein the vacuum tube is configured to apply negative pressure to the porous body as provided by the negative pressure source.

6. The medical system of claim 2, wherein the chamber is configured to receive the tube when the tube is in the second position, and at least a portion of the chamber is deformable to facilitate releasing the tube from the chamber.

7. The medical system of claim 2, wherein the chamber is configured to receive the tube when the tube is in the second position, and the chamber includes a slot that is configured to release the tube from the chamber.

8. The medical system of claim 1, wherein the cap assembly is removably mounted on an exterior surface of the distal end of the shaft.

9. The medical system of claim 8, wherein the cap assembly is selectively rotatable about the exterior surface of the distal end of the shaft such that the chamber is repositionable relative to the one or more channels.

10. The medical system of claim 1, wherein the porous body is a sponge, a gauze, a film, or a membrane.

11. The medical system of claim 1, wherein the chamber is at least partially transparent such that the porous body disposed inside the chamber is visible through the chamber.

12. The medical system of claim 1, wherein the chamber includes a window such that the porous body disposed inside the chamber is visible through the window by an imaging device.

13. The medical system of claim 12, wherein at least one of the one or more channels includes a working channel, and the window is aligned with the working channel to allow access to the working channel at the distal end of the shaft.

14. The medical system of claim 1, wherein the cap assembly includes a body with a pair of opposing halves, the body is configured to receive the distal end of the shaft and the pair of opposing halves is configured to grasp an exterior of the distal end to couple the cap assembly to the shaft.

15. The medical system of claim 14, wherein the cap assembly includes a fastener that is configured to couple the pair of opposing halves to one another, thereby securely attaching the cap assembly to the shaft.

16. A medical device, comprising:
a cap assembly configured to be attached about a shaft of an endoscope such that a working channel of the endoscope is accessible through an opening of the cap assembly at a distal end of the shaft, the cap assembly including a chamber that extends distally from the distal end; and
a porous body received within the chamber, the chamber configured to compress the porous body relative to the shaft;
wherein the porous body is movable relative to the cap assembly from a first position inside the chamber and in a compressed configuration to a second position outside of the chamber and in an expanded configuration.

17. The medical device of claim 16, further comprising a tube at least partially disposed inside the cap assembly, the tube is coupled to a negative pressure source at a first end and to the porous body at a second end, such that the negative pressure source is in fluid communication with the porous body via the tube;
wherein the tube is movable relative to the cap assembly to extend the porous body out from the chamber, and configured to generate a vacuum through the porous body in response to activating the negative pressure source.

18. The medical device of claim 17, wherein the chamber is configured to receive the tube when the tube extends the porous body out from the chamber, and at least a portion of the chamber is deformable to facilitate releasing the tube from the chamber.

19. The medical device of claim 17, wherein the chamber is configured to receive the tube when the tube extends the porous body out from the chamber, and the chamber includes a slot that is configured to release the tube from the chamber.

20. A method for treating a wound cavity with a medical device, comprising:
positioning a shaft of the medical device at the wound cavity, wherein a cap assembly is mounted onto the shaft such that the cap assembly is positioned adjacent to the wound cavity;
extending a porous body out from within the cap assembly in response to moving a tube relative to the cap assembly, such that the porous body expands upon exiting the cap assembly and entering the wound cavity, wherein the tube is coupled to the porous body and in fluid communication with a negative pressure source; and
applying a negative pressure at the wound cavity via the porous body in response to activating the negative pressure source.
